(19) United States
(12) Patent Application Publication
(10) Pub. No.: US 2025/0375102 A1
(43) Pub. Date: Dec. 11, 2025

(54) IMAGING UNIT OF ENDOSCOPE AND ENDOSCOPE

(71) Applicant: FUJIFILM Corporation, Tokyo (JP)

(72) Inventor: Hideyuki YAMADA, Ashigarakami-gun (JP)

(73) Assignee: FUJIFILM Corporation, Tokyo (JP)

(21) Appl. No.: 19/228,724

(22) Filed: Jun. 4, 2025

(30) Foreign Application Priority Data
Jun. 7, 2024 (JP) 2024-092817

(51) Int. Cl. A61B 1/05

(52) U.S. Cl. CPC A61B 1/05; A61B 2562/166

(57) ABSTRACT

An imaging unit of an endoscope includes: an imaging element; a circuit board to which the imaging element is connected; and a cable connected to the circuit board. The circuit board includes a first region in which the imaging element is disposed and that extends in a first direction, and a second region that extends in a second direction intersecting the first direction at an angle of less than 90 degrees, as viewed in a direction perpendicular to a light-receiving surface of the imaging element, and the cable is connected to the second region.

### CROSS-REFERENCE TO RELATED APPLICATIONS

This application is based on and claims priority under 35 USC 119 from Japanese Patent Application No. 2024-092817 filed on Jun. 7, 2024, the contents of which are incorporated herein by reference.

### BACKGROUND OF THE INVENTION

### 1. Field of the Invention

The technology of the present disclosure relates to an imaging unit of an endoscope and the endoscope.

### 2. Description of the Related Art

WO2014/203604A discloses an imaging unit for an endoscope configured by mounting an imaging element on a substrate.

JP2022-154577A discloses an endoscope comprising an image-capturing module including a lens barrel housing an optical system, an image sensor, a sensor holding member that relatively fixes the lens barrel and the image sensor, and a flexible substrate through which a signal of the image sensor is transmitted to a transmission cable, in which the flexible substrate is bendable at an arbitrary number of bending portions.

### SUMMARY OF THE INVENTION

An object of the technology of the present disclosure is to provide an imaging unit of an endoscope and the endoscope including the imaging unit.

An imaging unit of an endoscope according to an aspect of the technology of the present disclosure comprises an imaging element, a circuit board to which the imaging element is connected, and a cable connected to the circuit board, in which the circuit board includes a first region in which the imaging element is disposed and that extends in a first direction, and a second region that extends in a second direction intersecting the first direction at an angle of less than 90 degrees, as viewed in a direction perpendicular to a light-receiving surface of the imaging element, and the cable is connected to the second region.

According to the technology of the present disclosure, it is possible to provide an imaging unit of an endoscope and an endoscope comprising the imaging unit, which can increase a degree of freedom in disposition inside an insertion part.

### BRIEF DESCRIPTION OF THE DRAWINGS

FIG. 1 is a schematic view showing an endoscope 10 according to an embodiment of the technology of the present disclosure.

FIG. 2 is an enlarged perspective view showing the distal end part 30.

FIG. 3 is an assembly perspective view showing a configuration of the distal end part 30 shown in FIG. 2.

FIG. 4 is a schematic view showing an internal configuration of the distal end part 30 of the endoscope 10 shown in FIG. 1.

FIG. 5 is a perspective view of an imaging unit 200 shown in FIG. 4.

FIG. 6 is a perspective view of the imaging unit 200 shown in FIG. 4 as viewed from a direction different from that in FIG. 5.

FIG. 7 is a view of the imaging unit 200 shown in FIG. 4 as viewed in a direction of an optical axis 24AJ of an objective lens 24A.

FIG. 8 is a view in which a shape in which the circuit board 220 is unfolded is added to FIG. 4.

FIG. 9 is a view showing a modification example of the imaging unit 200 and is a view corresponding to FIG. 4.

FIG. 10 is a view of the imaging unit 200 shown in FIG. 9 as viewed in a direction of the optical axis 24AJ.

### DESCRIPTION OF THE PREFERRED EMBODIMENTS

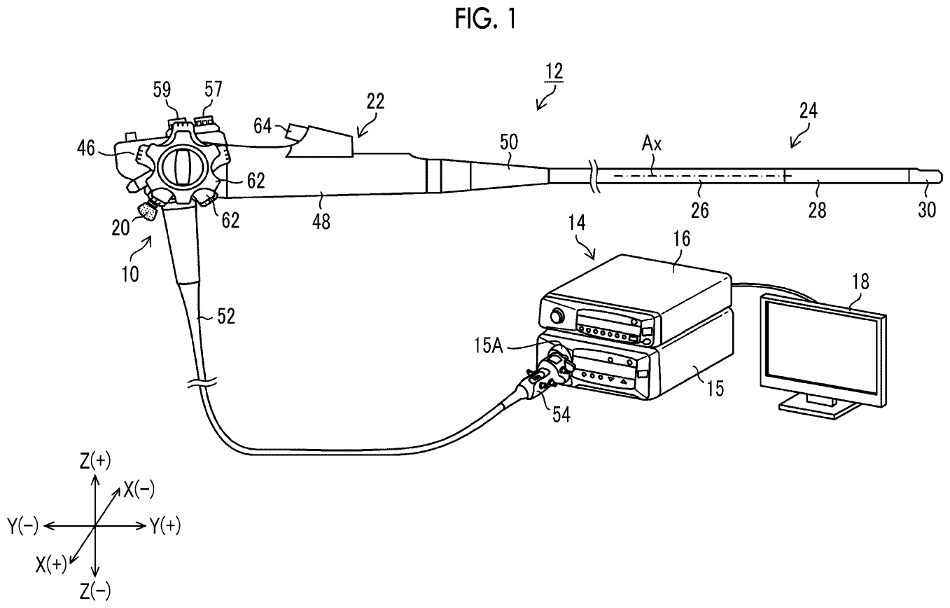

FIG. 1 is a configuration view of an endoscope system 12 comprising an endoscope 10 according to an aspect of the technology of the present disclosure. The endoscope system 12 comprises the endoscope 10, an endoscope processor apparatus 14, and a display 18.

The endoscope 10 is a side-viewing endoscope that is used as, for example, a duodenal endoscope. The endoscope 10 comprises an operation part 22 provided with an elevation operation lever 20, and an endoscope insertion part 24 that is connected to the operation part 22 and that is inserted into a subject to be examined.

The endoscope insertion part 24 is inserted into the subject to be examined through an oral cavity and is further inserted from an esophagus through a stomach to a duodenum. As a result, a prescribed examination or treatment, such as medical treatment, is performed on the duodenum using a treatment tool (not shown) inserted into the endoscope insertion part 24. Examples of the treatment tool include biopsy forceps of which a distal end part includes a cup capable of collecting a biological tissue, a knife for endoscopic sphincterotomy (EST), or a contrast tube.

The endoscope insertion part 24 extends along a longitudinal direction Ax from a proximal end side to a distal end side thereof and comprises a soft part 26, a bending part 28, and a distal end part 30 in this order from the proximal end side to the distal end side. A detailed configuration of the distal end part 30 will be described below, and an outline configuration of the distal end part 30 will be described first.

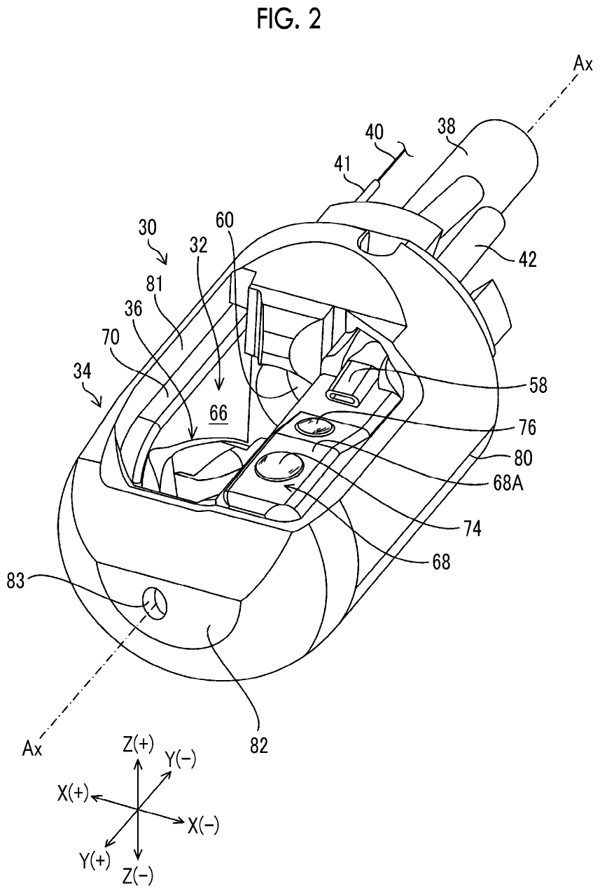

FIG. 2 is an enlarged perspective view showing the distal end part 30. Here, the endoscope 10 (see FIG. 1) of the embodiment is a side-viewing endoscope which is used as, for example, a duodenal endoscope, and the distal end part 30 of FIG. 2 has a configuration of the side-viewing endoscope.

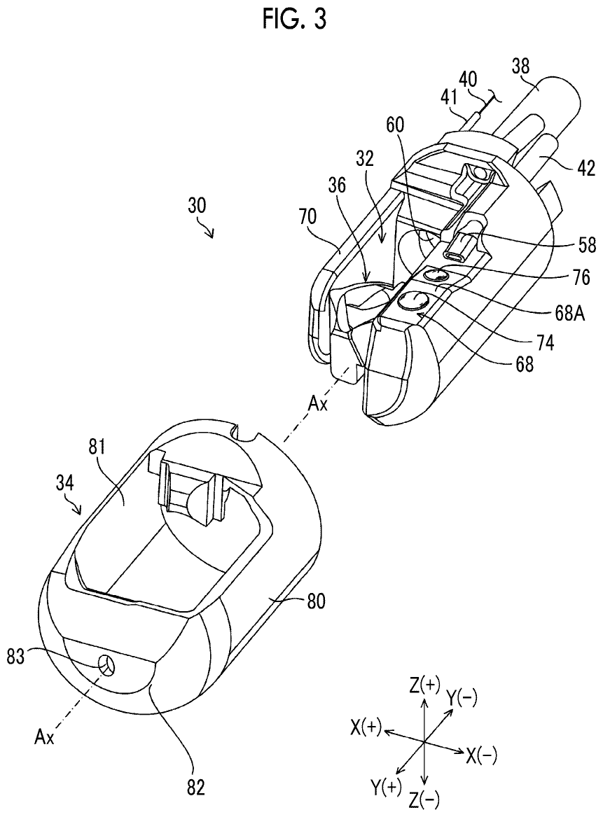

Further, FIG. 3 is an assembly perspective view showing a configuration of the distal end part 30 shown in FIG. 2. As shown in FIG. 3, the distal end part 30 includes a distal end part body 32 and a distal end cap 34 and is formed by mounting the distal end cap 34 on the distal end part body 32. The distal end part body 32 is provided on the distal end side of the endoscope insertion part 24 (see FIG. 1), and the distal end part body 32 is provided with an elevator 36 comprising a treatment tool guiding surface, which will be described below. FIGS. 2 and 3 show a state in which the elevator 36 is positioned at a reclined position.

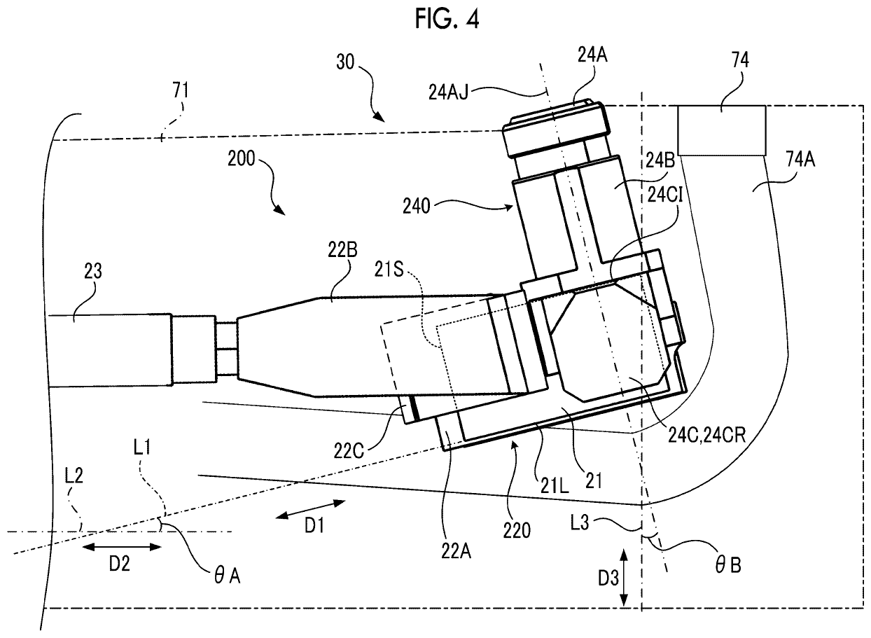

In addition, FIG. 2 and FIG. 3 show various contents disposed inside the endoscope insertion part 24 of the endoscope 10 (see FIG. 1). Specifically, a treatment tool channel 38 for guiding the distal end part of the treatment tool (not shown) to the distal end part body 32, an operation wire 40 for performing an operation to change a lead-out direction of the distal end part of the treatment tool to be led out of the distal end part body 32, and an air and water supply tube 42 are provided. In addition, although not shown in FIGS. 2 and 3, an angle wire for performing an operation to change a bending direction of the bending part 28 (see FIG. 1), a signal cable for transmitting an image signal, and a component such as a light guide 74A (see FIG. 4) for transmitting illumination light are provided.

In FIGS. 1 to 3, a description will be made using a three-dimensional Cartesian coordinate system having three directions (an X-axis direction, a Y-axis direction, and a Z-axis direction). For example, in FIGS. 1 to 3, a Z (+) direction indicates an upward direction, and a Z (−) direction indicates a downward direction. In addition, an X (+) direction indicates a rightward direction, and an X (−) direction indicates a leftward direction. Further, a Y (+) direction indicates a distal end side direction of the distal end part 30, and a Y (−) direction indicates a proximal end side direction of the distal end part 30. The Y (+) direction and the Y (−) direction are collectively referred to as a Y-axis direction. The Y-axis direction is parallel to the longitudinal direction Ax of the endoscope insertion part 24. The Z (+) direction and the Z (−) direction are collectively referred to as a Z-axis direction. The X (+) direction and the X (−) direction are collectively referred to as an X-axis direction.

Returning to FIG. 1, the operation part 22 is formed in a substantially cylindrical shape as a whole. The operation part 22 includes an operation part body 46 on which the elevation operation lever 20 is rotatably provided, and a grip portion 48 linked to the operation part body 46, and a proximal end part of the endoscope insertion part 24 is provided on a distal end side of the grip portion 48 via a bending prevention pipe 50. The grip portion 48 is a portion that is gripped by an operator during the operation of the endoscope 10.

A proximal end part of a universal cable 52 is connected to the operation part body 46. A connector device 54 is provided at a distal end part of the universal cable 52. The connector device 54 is connected to the endoscope processor apparatus 14.

The endoscope processor apparatus 14 comprises a light source device 15 and an image processing device 16. The light source device 15 comprises a processor-side connector 15A to which the connector device 54 is connected. In addition, the display 18 that displays an image that has been subjected to image processing by the image processing device 16 is connected to the image processing device 16.

The connector device 54 and the processor-side connector 15A transmit illumination light and, in a noncontact manner, power, image signals, and the like between the endoscope 10 and the endoscope processor apparatus 14 (wired transmission is also possible). As a result, illumination light from the light source device 15 is emitted from an illumination window 74 (see FIG. 2) provided on the distal end part body 32 through the light guide (an optical fiber cable, not shown). Further, an image signal of an image captured by an imaging unit (not shown) in an observation window 76 is subjected to image processing by the image processing device 16 and then is displayed as an image on the display 18.

The operation part body 46 is provided with an air and water supply button 57, a suction button 59, a pair of angle knobs 62, and the elevation operation lever 20.

The air and water supply button 57 is a button that can be pressed, and is connected to the air and water supply tube 42 and an air and water supply source (not shown). By operating the air and water supply button 57, air from the air and water supply source is ejected from an air and water supply nozzle 58 through the air and water supply tube 42. In addition, by pressing the air and water supply button 57, water from the air and water supply source is ejected from the air and water supply nozzle 58 through the air and water supply tube 42.

The suction button 59 is connected between the treatment tool channel 38 and a negative pressure source (not shown). In a case where the suction button 59 is pressed, air is suctioned from the suction port, which also serves as a treatment tool outlet port 60, through the treatment tool channel 38 by the negative pressure source. As a result, body fluids, such as blood, can be suctioned from the treatment tool outlet port 60 through the treatment tool channel 38. The suction port may be provided separately from the treatment tool outlet port 60.

The pair of angle knobs 62 are coaxially provided in the operation part body 46 so as to be rotationally movable. A proximal end part of each angle wire (not shown), which is opposite to a distal end part connected to the bending part 28, is connected to the pair of angle knobs 62. By rotationally moving each of the pair of angle knobs 62, each angle wire is pushed and pulled, so that the bending part 28 is bent up, down, left, and right.

The elevation operation lever 20 is provided on the operation part body 46 coaxially with the pair of angle knobs 62 so as to be rotationally movable and is rotationally moved by a hand of the operator who grips the grip portion 48. A proximal end part of the operation wire 40, which is opposite to a distal end part that is integrally molded with the elevator 36, is connected to the elevation operation lever 20 via a link mechanism (not shown). As a result, by rotationally moving the elevation operation lever 20, the operation wire 40 is pushed and pulled, so that an orientation of the elevator 36 is changed between the reclined position and an elevated position.

Next, a structure of the distal end part 30 shown in FIGS. 2 and 3 will be described in detail.

As described above, the distal end part 30 comprises the distal end part body 32 and the distal end cap 34 that is attachably and detachably mounted on the distal end part body 32. The distal end cap 34 is attached to the distal end part body 32 to define a space portion 66 at a position facing the treatment tool outlet port 60 functioning as the suction port, as will be described below.

The distal end part body 32 is made of a corrosion-resistant metal material. As shown in FIG. 3, the distal end part body 32 includes a pair of partition walls 68 and 70 that are projected toward a Y (+) direction side. The partition walls 68 and 70 are disposed to face each other in the X-axis direction. The partition wall 68 is disposed on an X (−) direction side and the partition wall 70 is disposed on an X (+) direction side as viewed from the Z (+) direction.

The illumination window 74 and the observation window 76 are disposed on an end surface 68A of the partition wall 68 facing the Z (+) direction side. The illumination window 74 and the observation window 76 are adjacent to each other in the Y-axis direction, and the illumination window 74 is disposed on the distal end side, and the observation window 76 is disposed on the proximal end side.

An emission end of the light guide (not shown) is disposed on an inner side of the illumination window 74. The illumination window 74 can illuminate the Z (+) direction side.

The imaging unit (not shown) is provided on an inner side of the observation window 76. The imaging unit images a subject present on the Z (+) direction side and the Y (−) direction side through the observation window 76. That is, the observation window 76 is provided to be inclined in the Y (−) direction with respect to the Z-axis direction so that the Z (+) direction (a lateral side of the distal end part 30) and the Y (−) direction side (a proximal end side of the distal end part 30) can be imaged. The imaging unit comprises, for example, an imaging optical system (not shown) and a complementary metal oxide semiconductor (CMOS) type or charge coupled device (CCD) type imaging element (not shown). An image signal of the subject, which is output from the imaging element, is input to the image processing device 16 through the signal cable (not shown), the connector device 54, and the processor-side connector 15A. As a result, the image of the subject is displayed on the display 18.

The air and water supply nozzle 58 is provided on the proximal end side with respect to the observation window 76 and at a position on the Z (+) direction side of the partition wall 68 of the distal end part body 32. The air and water supply nozzle 58 and the air and water supply tube 42 are connected to each other, and the air and water supply nozzle 58 jets air and water supplied from the air and water supply tube 42 toward the observation window 76.

The treatment tool outlet port 60 is provided on the proximal end side of the distal end part body 32. The treatment tool outlet port 60 is connected to the treatment tool channel 38, and the treatment tool outlet port 60 and the treatment tool channel 38 enable leading out of the treatment tool and suction of body fluids, such as blood.

The elevator 36 is disposed between the partition wall 68 and the partition wall 70 and in front of (on the distal end side of) the treatment tool outlet port 60. The elevator 36 is rotatably supported by a rotary shaft (not shown) parallel to the Y-axis. The operation wire 40 is inserted into a wire channel 41 and is connected to the elevator 36. By operating the operation wire 40, the elevator 36 is rotationally moved about the rotary shaft between the elevated position and the reclined position. The elevator 36 changes the orientation of the treatment tool led out of the treatment tool outlet port 60. The operation wire 40 is disposed on, for example, the partition wall 70 and is connected to the elevator 36 via the elevation operation lever (not shown). The elevator 36 is configured such that a distal end side thereof is movable in the Z-axis direction, and the Z-axis direction constitutes a movable direction of the elevator 36. In a case of being viewed in the longitudinal direction Ax, the X-axis direction is an intersection direction (specifically, a direction perpendicular to the movable direction) intersecting the movable direction. In a case of being viewed in the longitudinal direction Ax, the elevator 36 and the observation window 76 are provided at positions deviated from each other in the X-axis direction (intersection direction).

Next, an embodiment of the distal end cap 34 will be described. The distal end cap 34 has a substantially bottomed tubular shape in which the distal end side is closed and the proximal end side is open, and an internal space is formed. The distal end cap 34 is attachably and detachably attached to the distal end part body 32. In a case where the distal end cap 34 is attached to the distal end part body 32, the space portion 66 is defined at a position facing the treatment tool outlet port 60 (suction port). In the space portion 66, the elevator 36 is disposed between the pair of partition walls 68 and 70.

The distal end cap 34 comprises a sleeve portion 80, an opening portion 81 formed in the sleeve portion 80, a distal end surface portion 82, and a through-hole 83 provided in the distal end surface portion 82. The distal end cap 34 is made of an elastic material, for example, a rubber material such as fluororubber or silicone rubber, or a resin material such as polysulfone or polycarbonate.

The sleeve portion 80 has a substantially tubular shape and surrounds the treatment tool outlet port 60 and the partition walls 68 and 70. An opening of the sleeve portion 80 on the proximal end side is provided with a protrusion-like engaging portion (not shown) that engages with a groove-shaped engaged portion (not shown) formed on the distal end part body 32, and the engaging portion engages with the engaged portion, whereby the distal end cap 34 is attachably and detachably mounted on the distal end part body 32.

The opening portion 81 is an opening formed in a part of the sleeve portion 80 and allows the space portion 66 to be open to the outside. The opening portion 81 is open in the Z (+) direction in a case where the distal end part 30 is viewed from a position on the Z (+) direction side and exposes the space portion 66 and the end surface 68A of the partition wall 68. This makes it possible to lead out the treatment tool from the space portion 66 to the Z (+) direction side and to illuminate and image the subject.

The sleeve portion 80 is open through the opening portion 81, and the sleeve portion 80 does not need to be closed over an entire periphery. Meanwhile, it is preferable that a portion that engages with the distal end part body 32 has a closed annular shape.

The distal end surface portion 82 is connected to the sleeve portion 80 and is provided on the distal end side of the distal end cap 34 in the Y (+) direction. The distal end surface portion 82 covers a distal end surface of the distal end part body 32 on the Y (+) direction side. The distal end cap 34 is formed in a substantially bottomed tubular shape as a whole by the distal end surface portion 82 and the sleeve portion 80.

The through-hole 83 is provided in the distal end surface portion 82 of the distal end cap 34, which is a position different from the opening portion 81. The through-hole 83 allows the space portion 66 to communicate with the outside and functions as a negative pressure release portion that releases the negative pressure of the space portion 66. The through-hole 83 may be provided in the sleeve portion 80 as long as it is at a position different from the opening portion 81. In a case where the opening portion 81 is provided in the sleeve portion 80, it is preferable that the opening portion 81 is provided at a position that does not face the partition walls 68 and 70 accommodated in the space portion 66.

FIG. 4 is a schematic view showing a main part of an internal configuration of the distal end part 30 of the endoscope 10 shown in FIG. 1. FIG. 4 shows an exterior body 71 of the distal end part 30. Inside the exterior body 71, an imaging unit 200 including the imaging element 21, a circuit board 220 to which the imaging element 21 is connected, a cable 23 connected to the circuit board 220, and an imaging optical system 240 that collects subject light on the imaging element 21, and the illumination window 74 and the light guide 74A connected to the illumination window 74 are provided. FIG. 4 is a view showing a state as viewed from a direction perpendicular to a light-receiving surface of the imaging element 21. Although not shown in FIG. 4, the elevator 36 is provided on the front side of the paper surface with respect to the imaging unit 200, the illumination window 74, and the light guide 74A.

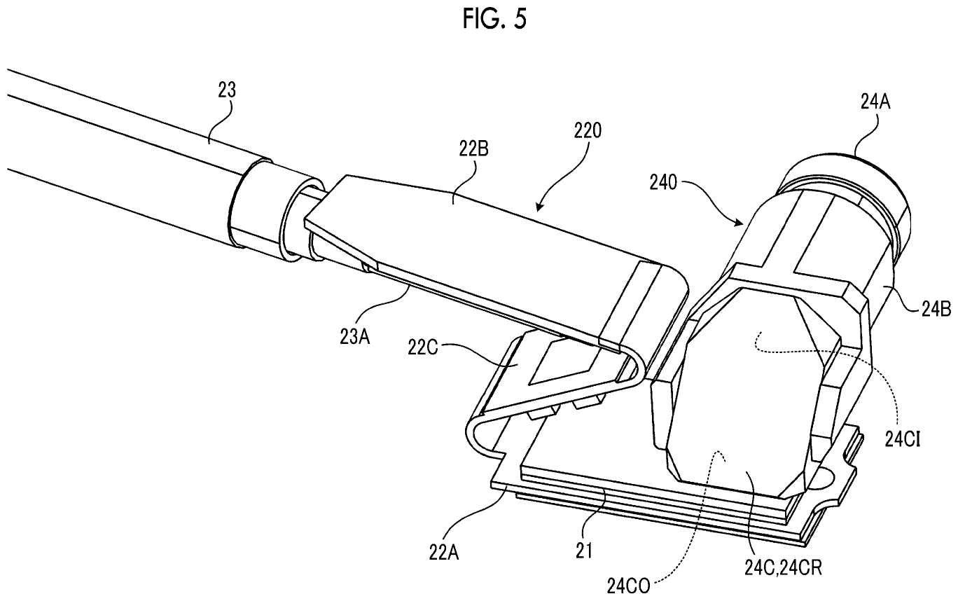

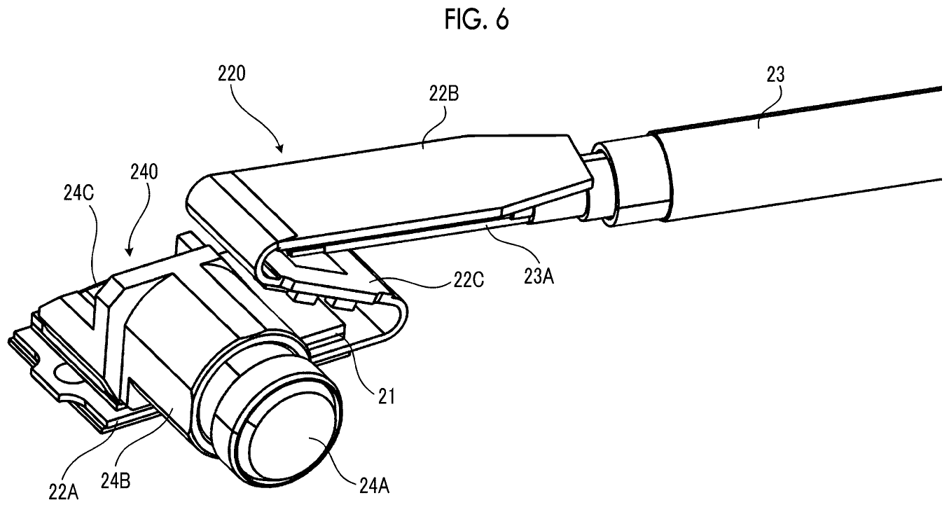

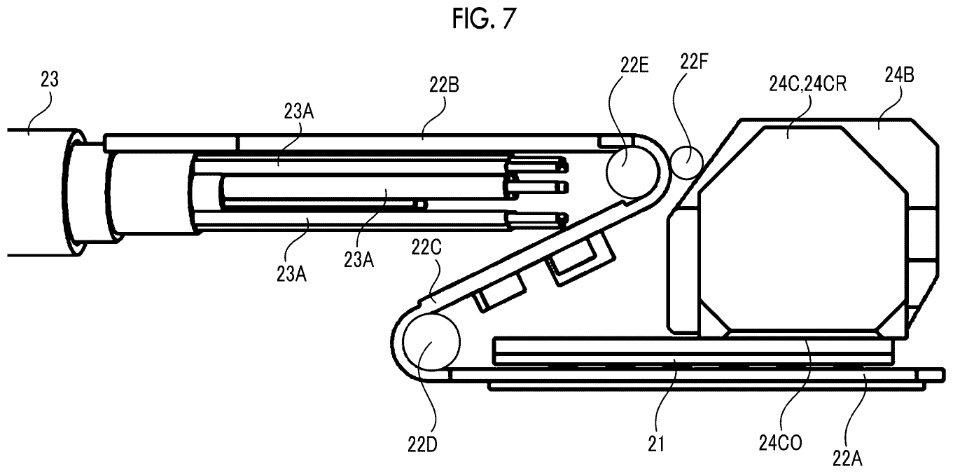

FIG. 5 is a perspective view of the imaging unit 200 shown in FIG. 4. FIG. 6 is a perspective view of the imaging unit 200 shown in FIG. 4 as viewed from a direction different from that in FIG. 5. FIG. 7 is a view of the imaging unit 200 shown in FIG. 4 as viewed in a direction of an optical axis 24AJ of an objective lens 24A.

The imaging optical system 240 includes a lens barrel 24B that holds a lens group including the objective lens 24A constituting the observation window 76, and a prism 24C supported at an end part of the lens barrel 24B on a side opposite to the objective lens 24A side. The prism 24C includes a light incident surface 24CI (see FIGS. 4 and 5) that is perpendicular to the light-receiving surface of the imaging element 21, a light-emitting inclined surface 24CO (see FIGS. 5 and 7) that is parallel to the light-receiving surface of the imaging element 21, and a reflecting surface 24CR (see FIGS. 4, 5, and 7) that reflects light incident on the light incident surface 24CI to the light-emitting inclined surface 24CO, and the light-emitting inclined surface 24CO is disposed to face the light-receiving surface of the imaging element 21. The light incident surface 24CI of the prism 24C faces a lens at an end of the lens group held by the lens barrel 24B on a side opposite to the objective lens 24A side. The light incident surface 24CI of the prism 24C is provided perpendicular to the optical axis 24AJ of the objective lens 24A. The direction perpendicular to the light-receiving surface of the imaging element 21 corresponds to the X-axis direction (intersection direction intersecting with the movable direction of the elevator 36) shown in FIGS. 2 and 3.

The imaging element 21 has a rectangular plate shape, and has a long side 21L and a short side 21S in a state shown in FIG. 4. The optical axis 24AJ of the objective lens 24A extends in a direction along the short side 21S of the imaging element 21. In other words, the short side 21S of the imaging element 21 and the optical axis 24AJ are parallel to each other, and the long side 21L of the imaging element 21 and the optical axis 24AJ are orthogonal to each other. FIG. 4 shows a first direction D1 along the long side 21L of the imaging element 21. A direction in which the optical axis 24AJ extends intersects a radial direction D3 (direction perpendicular to the longitudinal direction Ax, the same as the Z-axis direction in FIGS. 2 and 3) of the distal end part 30 at an angle of less than 90 degrees, and the optical axis 24AJ faces the proximal end side with respect to the radial direction D3. FIG. 4 shows a straight line L3 extending in the radial direction D3. It is preferable that an angle θB formed by the straight line L3 and the optical axis 24AJ (a first angle formed by the radial direction D3 and the direction in which the optical axis 24AJ extends) is 0 degrees or more and 20 degrees or less (more preferably 5 degrees or more and 15 degrees or less). In this way, the treatment tool that is led out from the treatment tool outlet port 60 and is lifted by the elevator 36 can be appropriately imaged.

The circuit board 220 comprises a first region 22A in which the imaging element 21 is disposed, a second region 22B to which the cable 23 is connected, and a third region 22C between the first region 22A and the second region 22B. In the state shown in FIG. 4, the first region 22A is provided on the side opposite to the elevator 36 side (the back side of the paper surface) with respect to the second region 22B and the third region 22C. It is preferable that the circuit board 220 has flexibility, and in the present embodiment, the circuit board 220 is configured by being folded back at a plurality of locations using the flexibility. As shown in FIG. 7, the circuit board 220 has a substantially Z-shape in a direction of the optical axis 24AJ.

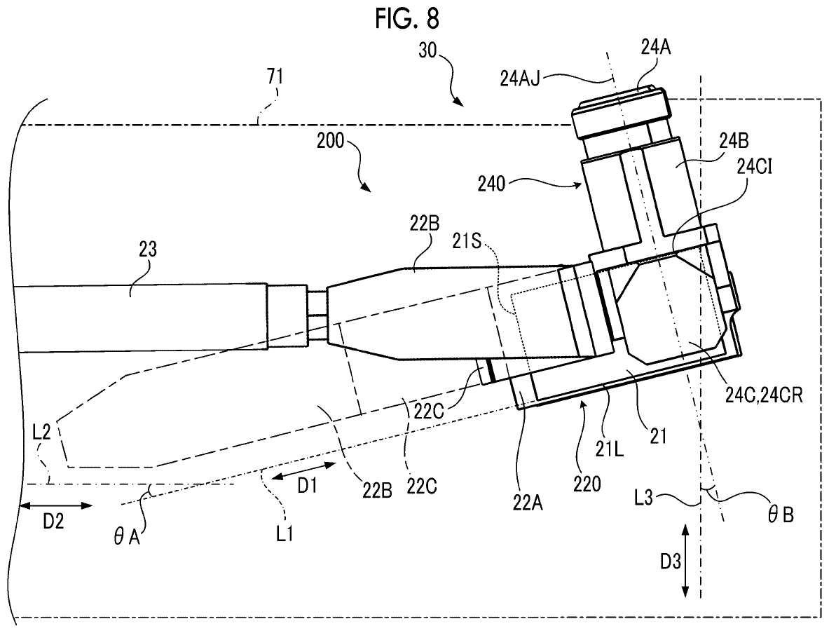

FIG. 8 is a view in which a shape in which the circuit board 220 is unfolded is added to FIG. 4. In FIG. 8, the illumination window 74 and the light guide 74A are not shown. As shown in FIG. 8, in the unfolded state of the circuit board 220 in which the second region 22B and the third region 22C are not folded back, the first region 22A, the third region 22C, and the second region 22B are arranged linearly in this order. That is, the unfolded shape of the circuit board 220 is a linear shape extending along the first direction D1.

The first region 22A forms a rectangular plate shape extending in the first direction D1. The thickness direction of the first region 22A coincides with a direction perpendicular to the light-receiving surface of the imaging element 21.

As shown in FIG. 8, the third region 22C forms a rectangular plate shape extending from one end of the first region 22A on the proximal end side in the first direction D1. As shown in FIGS. 5 and 6, the third region 22C is folded back to the other end side (distal end side) of the first region 22A in the first direction D1 at the end portion on the first region 22A side. The third region 22C constitutes a first folded portion.

As shown in FIG. 8, the second region 22B forms a rectangular plate shape extending from an end edge of the third region 22C on a side opposite to the first region 22A side, and is folded back to the proximal end side of the first region 22A in the first direction D1 at the end edge. The second region 22B constitutes a second folded portion. The second region 22B and the third region 22C constitute a folded portion that is folded back from one end of the first region 22A in the first direction D1.

The third region 22C is folded back along the first direction D1 at a boundary portion with the first region 22A, whereas the second region 22B is folded back along the second direction D2 that intersects the first direction D1 at a boundary portion with the third region 22C. That is, the first region 22A and the third region 22C extend in the first direction D1, whereas the second region 22B extends in the second direction D2. A long cable 23 that extends to the universal cable 52 of the endoscope 10 is connected to the second region 22B.

As shown in FIG. 7, the cable 23 has a configuration in which a plurality of signal lines 23A are bundled. Each signal line 23A is exposed in a region between the second region 22B and the third region 22C on the distal end side of the cable 23, and is electrically and mechanically connected to a terminal provided on the circuit board 220. The cable 23 extends along the second direction D2 from the second region 22B and is provided up to the universal cable 52. That is, the extending direction of the cable 23 is configured to intersect with the first direction D1. From the viewpoint of improving the space efficiency inside the endoscope insertion part 24, it is preferable that the cable 23 is in a state close to being parallel to the longitudinal direction Ax. For example, it is preferable that the angle formed by the second direction D2 and the longitudinal direction Ax is 0 degrees or more and 5 degrees or less.

FIG. 4 shows a straight line L2 extending in the second direction D2 and a straight line L1 extending in the first direction D1, and an angle θA formed by the straight line L1 and the straight line L2 (the same as the angle formed by the first direction D1 and the second direction D2) is more than 0 degrees and less than 90 degrees. The angle θA is set to, for example, a range of about ±2 degrees of the angle θB, so that the cable 23 can be brought into a state close to being parallel to the longitudinal direction Ax. In addition, by setting the angle θB to be 0 degrees or more and 20 degrees or less (more preferably 5 degrees or more and 15 degrees or less) and setting the difference between the angle θA and the angle θB to be ±5 degrees or less (more preferably ±3 degrees or less), the cable 23 can be brought into a state close to being parallel to the longitudinal direction Ax, and the diameter of the endoscope insertion part 24 can be reduced.

Since the circuit board 220 configured as described above has flexibility, it is easy to configure the second region 22B along the second direction D2. For example, the linear circuit board 220 in the unfolded state is folded back along the first direction D1 at the boundary portion between the third region 22C and the first region 22A, an adhesive 22D (see FIG. 7) is applied to the boundary portion, or the like, the first region 22A and the third region 22C are adhered to each other, and the folded-back state is maintained.

Next, the circuit board 220 is folded back along the first direction D1 at the boundary portion between the third region 22C and the second region 22B to make the second region 22B and the first region 22A substantially parallel to each other, and then the second region 22B is slid in a direction in which the second region 22B approaches the objective lens 24A (moved in a direction along the surface of the second region 22B) with the boundary portion as a fulcrum, the adhesive 22E (see FIG. 7) is applied to the boundary portion, or the like, the second region 22B and the third region 22C are adhered to each other, and the folded-back state is maintained.

In this way, the circuit board 220 having the shape shown in FIGS. 4 to 7 can be manufactured. As shown in FIG. 7, it is preferable that the circuit board 220 is further adhered to the lens barrel 24B with the adhesive 22F at the boundary portion between the second region 22B and the third region 22C. The adhesive 22E is applied to the proximal end part of the second region 22B, but the proximal end part overlaps with another region of the circuit board 220 other than the second region 22B (specifically, an end part of the third region 22C) in FIG. 4. By adhering the proximal end part and the other region, the extending direction of the second region 22B is maintained.

With the structure of the distal end part 30, the optical axis 24AJ can be directed to the lateral side (radial direction D3) and the proximal end side of the distal end part 30 while the cable 23 is made substantially parallel to the longitudinal direction Ax. Therefore, it is not necessary to complicate the routing of the cable 23, and the degree of freedom in disposition of the imaging unit 200 inside the endoscope insertion part 24 can be increased. In addition, since the circuit board 220 has a linear shape in the unfolded state, the circuit board and the components used in the existing endoscope can be shared, and the manufacturing cost of the imaging unit 200 can be reduced. In addition, since the orientation in which the second region 22B extends is adjusted by using the flexibility of the circuit board 220, it is possible to easily manufacture the imaging unit 200. In addition, since the orientation in which the second region 22B extends can be finely adjusted, the orientation of the second region 22B can be made appropriate for each individual. In addition, the first region 22A of the circuit board 220 on which the imaging element 21 is mounted is provided on the side opposite to the elevator 36 side with respect to the second region 22B and the third region 22C. Therefore, it is possible to prevent the imaging element 21 from coming into contact with the portion having high rigidity in which the elevator 36 is accommodated.

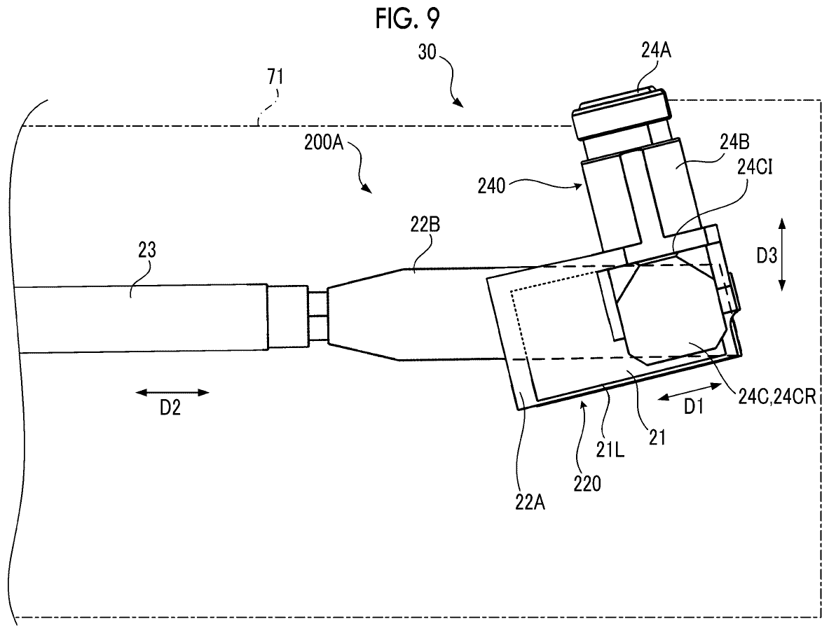

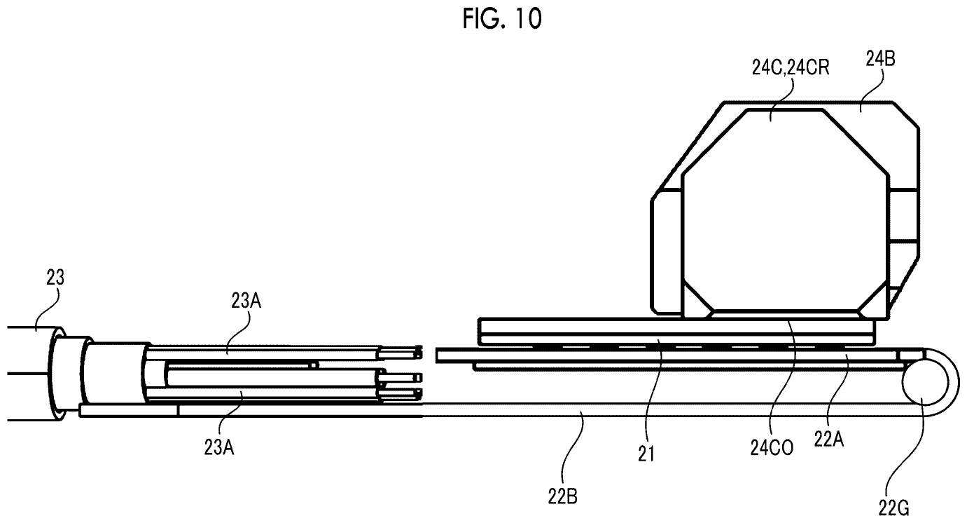

In the above description, the circuit board 220 is configured to be folded back at two locations, but the present invention is not limited thereto. For example, the circuit board 220 may have a configuration in which the circuit board 220 is folded back only at one location. FIG. 9 is a view showing a modification example of the imaging unit 200 and is a view corresponding to FIG. 4. FIG. 10 is a view of the imaging unit 200 shown in FIG. 9 as viewed in the direction of the optical axis 24AJ.

In the imaging unit 200A shown in FIG. 9, the third region 22C of the circuit board 220 in the imaging unit 200 is removed, and the second region 22B is folded back from the other end of the first region 22A in the first direction D1 to the proximal end side and extends in the second direction D2. As shown in FIG. 10, since the proximal end part of the second region 22B overlaps with the first region 22A, the bending state of the second region 22B with respect to the first region 22A is maintained by applying the adhesive 22G to the overlapping region. The cable 23 is connected to the distal end part of the second region 22B and extends in the second direction D2. In the imaging unit 200A shown in FIGS. 9 and 10, the second region 22B constitutes a folded portion.

Even in the imaging unit 200A shown in FIGS. 9 and 10, the same effects as the imaging unit 200 shown in FIGS. 4 to 8 can be obtained.

As described above, at least the following matters are described in the present specification.

(1)

An imaging unit of an endoscope including:

an imaging element;

a circuit board to which the imaging element is connected; and

a cable connected to the circuit board,

in which the circuit board includes a first region in which the imaging element is disposed and that extends in a first direction, and a second region that extends in a second direction intersecting the first direction at an angle of less than 90 degrees, as viewed in a direction perpendicular to a light-receiving surface of the imaging element, and

the cable is connected to the second region.

(2)

The imaging unit according to (1),

in which the first direction and an extending direction of the cable from the second region intersect each other.

(3)

The imaging unit according to (1) or (2),

in which the first direction is a direction along a long side of the imaging element.

(4)

The imaging unit according to (3),

in which the circuit board has flexibility.

(5)

The imaging unit according to (4),

in which the circuit board has a folded portion that is folded back from one end of the first region in the first direction, and

at least a part of the folded portion constitutes the second region.

(6)

The imaging unit according to (5),

in which the folded portion includes a first folded portion that is folded back from the one end to the other end side of the first region in the first direction, and a second folded portion that is folded back from the first folded portion to one end side of the first region, and

the second folded portion constitutes the second region.

(7)

The imaging unit according to (5) or (6),

in which a shape of the circuit board in a state of being unfolded is a linear shape extending in the first direction.

(8)

The imaging unit according to (7),

in which, as viewed in the direction perpendicular to the light-receiving surface of the imaging element, a proximal end part of the second region overlaps with another region of the circuit board, and the proximal end part and the other region are adhered to each other.

(9)

An endoscope including:

an endoscope insertion part including the imaging unit according to any one of (1) to (8) at a distal end part of the endoscope insertion part,

in which the imaging unit includes an imaging optical system that collects subject light on the imaging element,

the distal end part is provided with an elevator for a treatment tool,

an objective lens of the imaging optical system is provided toward a lateral side and a proximal end side of the distal end part, and

the objective lens and the elevator are provided at positions deviated from each other in an intersection direction intersecting a movable direction of the elevator as viewed in a longitudinal direction of the endoscope insertion part.

(10)

The endoscope according to (9),

in which the light-receiving surface of the imaging element is provided in the intersection direction.

(11)

The endoscope according to (10),

in which the first region of the circuit board is provided on a side opposite to an elevator side with respect to the second region as viewed in a direction perpendicular to the light-receiving surface.

(12)

The endoscope according to any one of (9) to (11),

in which an optical axis of the objective lens extends in a direction along a short side of the imaging element, and

as viewed in a direction perpendicular to the light-receiving surface, a first angle formed by a radial direction of the distal end part and a direction in which the optical axis extends is 5 degrees or more and 15 degrees or less.

(13)

The endoscope according to (12),

in which a difference between an angle between the first direction and the second direction and the first angle is ±5 degrees or less.

### Explanation of References

10: endoscope
12: endoscope system
14: endoscope processor apparatus
15: light source device
15A: processor-side connector
16: image processing device
18: display
20: elevation operation lever
26: soft part
28: bending part
30: distal end part
32: distal end part body
34: distal end cap
36: elevator
38: treatment tool channel
40: operation wire
41: wire channel
42: air and water supply tube
46: operation part body
48: grip portion
50: pipe
52: universal cable
54: connector device
57: air and water supply button
58: air and water supply nozzle
59: suction button
60: treatment tool outlet port
62: angle knob
66: space portion
68, 70: partition wall
68A: end surface
74: illumination window
76: observation window
80: sleeve portion
81: opening portion
82: distal end surface portion
83: through-hole
200, 200A: imaging unit
21: imaging element
21L: long side
21S: short side
220: circuit board
22A: first region
22B: second region
22C: third region
22D, 22E, 22F, 22G: adhesive
23: cable
23A: signal line
240: imaging optical system
24A: objective lens
24B: lens barrel
24C: prism
24CI: light incident surface
24CO: light-emitting inclined surface
24CR: reflecting surface
71: exterior body
24AJ: optical axis
D1: first direction
D2: second direction
D3: radial direction
L1, L2, L3: straight line

What is claimed is:

1. An imaging unit of an endoscope comprising:
an imaging element;
a circuit board to which the imaging element is connected; and
a cable connected to the circuit board,
wherein the circuit board includes a first region in which the imaging element is disposed and that extends in a first direction, and a second region that extends in a second direction intersecting the first direction at an angle of less than 90 degrees, as viewed in a direction perpendicular to a light-receiving surface of the imaging element, and
the cable is connected to the second region.

2. The imaging unit according to claim 1,
wherein the first direction and an extending direction of the cable from the second region intersect each other.

3. The imaging unit according to claim 2,
wherein the first direction is a direction along a long side of the imaging element.

4. The imaging unit according to claim 3,
wherein the circuit board has flexibility.

5. The imaging unit according to claim 4,
wherein the circuit board has a folded portion that is folded back from one end of the first region in the first direction, and
at least a part of the folded portion constitutes the second region.

6. The imaging unit according to claim 5,
wherein the folded portion includes a first folded portion that is folded back from the one end to the other end side of the first region in the first direction, and a second folded portion that is folded back from the first folded portion to one end side of the first region, and
the second folded portion constitutes the second region.

7. The imaging unit according to claim 6,
wherein a shape of the circuit board in a state of being unfolded is a linear shape extending in the first direction.

8. The imaging unit according to claim 7,
wherein, as viewed in the direction perpendicular to the light-receiving surface of the imaging element, a proximal end part of the second region overlaps with another region of the circuit board, and the proximal end part and the other region are adhered to each other.

9. An endoscope comprising:
an endoscope insertion part including the imaging unit according to claim 1 at a distal end part of the endoscope insertion part,
wherein the imaging unit includes an imaging optical system that collects subject light on the imaging element,
the distal end part is provided with an elevator for a treatment tool,
an objective lens of the imaging optical system is provided toward a lateral side and a proximal end side of the distal end part, and
the objective lens and the elevator are provided at positions deviated from each other in an intersection direction intersecting a movable direction of the elevator as viewed in a longitudinal direction of the endoscope insertion part.

10. The endoscope according to claim 9,
wherein the light-receiving surface of the imaging element is provided in the intersection direction.

11. The endoscope according to claim 10,
wherein the first region of the circuit board is provided on a side opposite to an elevator side with respect to the second region as viewed in a direction perpendicular to the light-receiving surface.

12. The endoscope according to claim 9,
wherein an optical axis of the objective lens extends in a direction along a short side of the imaging element, and
as viewed in a direction perpendicular to the light-receiving surface, a first angle formed by a radial direction of the distal end part and a direction in which the optical axis extends is 5 degrees or more and 15 degrees or less.

13. The endoscope according to claim 12,
wherein a difference between an angle between the first direction and the second direction and the first angle is ±5 degrees or less.
